(19) United States
(12) Patent Application Publication
(10) Pub. No.: US 2018/0028591 A1
(43) Pub. Date: Feb. 1, 2018

(54) MALE AND FEMALE INFERTILITY HERBAL COMPOSITION

(71) Applicant: Shaobin Ke, Temple City, CA (US)

(72) Inventor: Shaobin Ke, Temple City, CA (US)

(21) Appl. No.: 15/224,991

(22) Filed: Aug. 1, 2016

(51) Int. Cl. A61K 36/9066; A61K 36/537; A61K 36/752; A61K 36/70; A61K 36/296; A61K 36/185; A61K 36/73; A61K 36/481; A61K 36/8945; A61K 36/65; A61K 36/28; A61K 36/605; A61K 36/39; A61K 36/344

(52) U.S. Cl. CPC A61K 36/9066; A61K 36/39; A61K 36/537; A61K 36/752; A61K 36/70; A61K 36/296; A61K 36/344; A61K 36/73; A61K 36/481; A61K 36/8945; A61K 36/65; A61K 36/28; A61K 36/605; A61K 36/185

(57) ABSTRACT

An herbal composition that enhances and promotes fertility in both males and females by nourishing the kidney, regulating the liver and spleen, improving circulation of Qi and blood, and balancing the Yin and Yang through a unique composition consisting of natural herbs known in traditional Chinese medicine. The herbs create a synergy between each other that works to enhance fertility for both males and females. The composition consists of the following herbal ingredients mixed in varying weight percentages. goji berry, dodder seed, red sage root, green orange peel, processed fleece flower root, horny goat weed, codonopsis, raspberry, astragalus root, Chinese yam, white peony root, false daisy, mulberry, curcuma tuber, and cynomorium. The herbal ingredients are cleaned, purified and/or processed prior to formulation. The formulation process is at least partially dependent on the desired means of preparation and administering the composition.

### FIELD OF THE INVENTION

The present invention relates generally to a male and female infertility herbal composition. More so, the present invention relates to an herbal composition that enhances and promotes fertility in both males and females by nourishing the kidney, regulating the liver and spleen, improving circulation of Qi and blood, and balancing the Yin and Yang through a unique composition consisting of the following herbs in varying weight percentages including: Fructus Lycii, Semen Cuscutae, Radix Salviae Miltiorrhizae, Pericarpium Citri Reticulatae Viride preparata, Radix Polygoni Multiflori preparata, Herba Epimedii, Radix Codonopsis, Fructus Rubi, Radix Astragali, Rhizoma Dioscoreae, Radix Paeoniae Alba, Herba Ecliptae, Fructus Mori, Radix Curcumae, and Herba Cynomorii.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It is known that infertility is the inability of a person, animal or plant to reproduce by natural means. Infertility is usually not the natural state of a healthy adult organism. Infertility may describe a woman who is unable to conceive as well as being unable to carry a pregnancy to full term. There are many biological and other causes of infertility, including some that medical intervention can treat. Additionally, an estimated six percent of adult males are thought to be infertile.

Infertility is more specifically defined as the inability to achieve a pregnancy after one year of unprotected intercourse. Conception is normally achieved within 12 months in 80-85 percent of couples using nocontraceptive measures; thus an estimated 15 percent of couples attempting their first pregnancy will have difficulty conceiving. While certain cases of male infertility are due to anatomical abnormalities such as varicoceles, ductal obstructions, or ejaculatory disorders, an estimated 40-90 percent of cases are due to deficient sperm production of unidentifiable origin. Sinclair, S, 5(1) Alt Med Review 28-38 (2000).

Often, medical treatment of infertility generally involves the use of fertility medication, medical device, surgery, or a combination of the following. If the sperm are of good quality and the mechanics of the woman's reproductive structures are good (patent fallopian tubes, no adhesions or scarring), a course of ovarian stimulating medication maybe used. The physician or WHNP may also suggest using a conception cap cervical cap, which the patient uses at home by placing the sperm inside the cap and putting the conception device on the cervix, or intrauterine insemination (IUI), in which the doctor or WHNP introduces sperm into the uterus during ovulation, via a catheter. In these methods, fertilization occurs inside the body.

According to information from American pregnancy Association, female infertility factors contribute to approximately 50% of all infertility cases, and female infertility alone accounts for approximately one-third of all infertility cases. The main causes of female infertility include the abnormal function of uterus and ovary, as well as endocrine disorders. These can be resulted from tubal obstruction, immune infertility, psychological infertility, anovulation, luteal dysfunction, annex inflammation, amenorrhea, irregular menstruation, poor uterine development and other gynecological illnesses.

In many instances, abnormal sperm, no sperm (azoospermia), and inadequate numbers of sperm (oligospermia), or problems with ejaculation are main reasons leading to male infertility. They can be caused by hypothalamic or pituitary disorder, gonad disorder, sperm transport disorder, or unknown reason (40-50%). Anxiety and depression are becoming one main cause of infertility to both female and male in modern society.

Even though many treatment methods, such as hormone treatment, surgery, in vitro fertilization (IVF), intrauterine insemination (IUI), are available for female and male infertility, many women can still not get pregnant.

It is widely recognized that traditional Chinese medicine teaches that die kidney is the congenital base of life. The definition of kidney in traditional Chinese medicine is different from kidney organ in Western medicine. The kidney in traditional Chinese medicine is considered as a functional unit of controlling reproduction and growth. Modern research has demonstrated that the kidney functions cover the regulation of core physiological axis of hypothalamus-pituitary, including the hypothalamic-pituitary-gonadal axis (HPG axis). Kidney is the main focus in traditional Chinese medicine treatment of both female and male infertility. Functions of liver and spleen in traditional Chinese medicine also impact male and female fertility. In addition, poor circulation and imbalance of Yin and Yang also affect the sexual function and fertility of both male and female.

Further, the decline of sexual function is part of the signs of aging. Traditional Chinese medicine believes that aging starts with decline of kidney function. Strengthening the function of “ kidney” is one of the important strategies of anti-aging and treatment for infertility in traditional Chinese medicine. Regulating the function of liver and spleen, nourishing Yin and Yang, and improving circulation of Qi and blood are often combined in treatment of infertility based on traditional Chinese medicine diagnosis and differentiation.

Other proposals have involved fertility compositions. The problem with these compositions is that they do not provide natural ingredients that create a synergy to enhance fertility rates for both men and women. Even though the above cited compositions meets some of the needs of the market, an herbal composition that enhances and promotes fertility in both males and females by nourishing the kidney, regulating the liver and spleen, improving circulation of Qi and blood, and balancing the Yin and Yang through a unique composition consisting of natural herbal ingredients is still desired.

### SUMMARY

Illustrative embodiments of the disclosure are generally directed to an herbal composition that enhances and promotes fertility in both males and females by nourishing the kidney, regulating the liver and spleen, improving circulation of Qi and blood, and balancing the Yin and Yang through a unique composition consisting of natural herbs known in traditional Chinese medicine. The herbs create a synergy between each other that works to enhance fertility for both males and females.

Specifically, the herbal ingredients that comprise the composition are efficacious for regulating function of the hypothalamic-pituitary-gonadal axis (HPG axis). Further, the ingredients, when mixed together, create activities of anti-inflammation, anti-oxidation, anti-pathogenic microorganisms, and are able to improve microcirculation. This results in enhanced gonadal function to improve fertility.

The composition consists of the following herbal ingredients mixed in varying weight percentages: Fructus Lycii, Semen Cuscutae, Radix Salviae Miltiorrhizae, Pericarpium Citri Reticulatae Viride preparata, Radix Polygoni Multiflori preparata, Herba Epimedii, Radix Codonopsis, Fructus Rubi, Radix Astragali, Rhizoma Dioscoreae, Radix Paeoniae Alba, Herba Ecliptae, Fructus Mori, Radix Curcumae, and Herba Cynomorii. The herbal ingredients may be mechanically processed prior to mixing. The mixing process is at least partially dependent on the desired means of administering the composition.

There are various embodiments of weight percentages for the present disclosure. The following ingredients reference the allowable weight percentage range used to formulate the composition: Fructus Lycii (3-15%), Semen Cuscutae (3-15%), Radix Salviae Miltiorrhizae (3%-10%), Pericarpium Citri Reticulatae Viride preparata (1-5%), Radix Polygoni Multiflori preparata (3-15%), Herba Epimedii (3-10%), Radix Codonopsis (3-15%), Fructus Rubi (3-15%), Radix Astragali (3-10%), Rhizoma Dioscoreae (3-10%), Radix Paeoniae Alba (3-10%), Herba Ecliptae (3-10%), Fructus Mori (5-10%), Radix Curcumae (3-10%), and Herba Cynomorii (3-10%).

In some embodiments, the composition may be administered in at least one of the following ways: a pill, a dripping pill, a tablet, an orally disintegrating tablet, a granule, a powder, a lozenge, a capsule, a cream, a suppository, and an oral liquid.

The safety of the composition was tested in laboratories with rats and standard scientific procedure known in the art. An acute toxicity experiment and a sub-chronic toxicity experiment showed negligible amounts of toxicity, indicating the composition is safe for human consumption.

In one aspect, an herbal composition for enhancing fertility in males and females, consists of:

about 3 to 15 weight percent Fructus Lycii, about 3 to 15 weight percent Semen Cuscutae, about 3 to 10 weight percent Radix Salviae Miltiorrhizae, about 1 to 5 weight percent Pericarpium Citri Reticulatae Viride preparata, about 3 to 15 weight percent Radix Polygoni Multiflori preparata, about 3 to 10 weight percent Herba Epimedii, about 3 to 15 weight percent Radix Codonopsis , about 3 to 15 weight percent Fructus Rubi, about 3 to 10 weight percent Radix Astragali, about 3 to 10 weight percent Rhizoma Dioscoreae, about 3 to 10 weight percent Radix Paeoniae Alba, about 3 to 10 weight percent Herba Ecliptae, about 5 to 10 weight percent Fructus Mori, about 3 to 10 weight percent Radix Curcumae, and about 3 to 10 weight percent Herba Cynomorii.

In another aspect, the composition is administered in at least one of the following ways: oral, topical, suppository, intravenous, intradermic, intragaster, intramuscular, and intraperitoneal.

In another aspect, the composition includes at least one member selected from the group consisting of: a tablet, an orally disintegrating tablet, a pill, a dripping pill, a lozenge, a capsule, a cream, a granule, a suppository, and an oral liquid.

In another aspect, the composition is administered in a dosage ranging from 0.01 to 500 miligrams per kilogram of body weight.

In another aspect, the composition consists of a plurality of herbal ingredients prepared through processing methods, extraction methods, and purification methods.

In another aspect, the processing methods include at least one member selected from the group consisting of: washing, steaming, extracting, roasting, herb frying, salt frying, honey frying, wine frying, earth frying, vinegar frying, calcining drying, heating, and grinding.

In another aspect, the extraction methods include at least one member selected from the group consisting of: soaking, heating, steaming, evaporation, compressing, and solvent extraction with at least one of the following: water, alcoholic solvents mixed with water, petroleum ether, hexane, diethyl amine, diethyl ether, cyclohexane, tert-butyl alcohol, isopropanol, Acetonitrile, aceton, ethanol, methyl isobutyl ketone, isobutyl alcohol, 1-propanol, methyl ethyl ketone, 2-butanol, isoamyl alcohol, 1-butanol, diethyl ketone, 1-octanol, p-xylene, m-xylene, toluene, dimethoxyethane, benzene, butyl acetate, 1-chlorobutane, tetrahydrofuran, ethyl acetate, o-xylene, hexamethylphosphorus triamide, 2-ethoxyethyl ether. N,N-dimethylacetamide, diethylene glycol dimethyl ether, N,N-dimethylformamide, 2-methoxyethanol, pyridine, propanoic acid, 2-methoxyethyl acetate, benzonitrile, 1-Methyl-2-pyrrolidinone, hexamethylphosphoramide, 1,4-dioxane, acetic acid, acetic anhydride, dimethyl sulfoxide, chlorobenzene, ethylene glycol, diethylene glycol, propylene carbonate, formic acid, 1,2-dichloroethane, glycerin, carbon disulfide, 1,2-dichlorobenzene, methylene chloride, nitromethane, 2,2,2-trifluoroethanol, chloroform, 1,1,2-trichlorotrifluoroethane, carbon tetrachloride, and tetrachloroethylene.

In another aspect, the purification methods include at least one member selected from the group consisting of: column separation, solvent partition, evaporation, and solvent precipitation.

In another aspect, Fructus Lycii, goji berry, is the fruit of the plant Lycium barbarum L.; Semen Cuscutae, dodder seed, is the seed of the plant Cuscuta chinensis Lam.; Radix Salviae Miltiorrhizae, red sage root, is the root and rhizome of the plant Salvia miltiorrhiza Bge.; Pericarpium Citri Reticulatae Viride preparata, green orange peel, unripe tangerine peel, or immature mandarin orange peel, is the vinegar processed green rind of immature fruit of the plant Citrus reticulate Blanco.; Radix Polygoni Multiflori preparata, processed fleece flower root, or Chinese knotweed, is the processed root of the plant Polygonum multiflorum Thunb.; Herba Epimedii, horny goat weed, is the arial part of the plant Epimedium brevicornum Maxim., Epimedium sagittatum (Sieb. Et Zucc.) Maxim, Epimedium pubescens Maxim, or Epimedium wushanense T. S. Ying, Epimedium koreanum Nakai.; Radix Codonopsis, codonopsis root, is the root of the plant Codonopsis pilosula (Franch) Nannf., Codonopsis pilosula Nannf. var. moderta (Nannf.) L., or Codonopsis tangshen Oliv.; Fructus Rubi, raspberry, is the fruit of the plant Rubus chingii Hu.; Radix Astragali, astragalus root, is the root of the plant Astragalus membranaceus (Fisch.) Bge. var. mongholicus (Bge.) Hsiao, Astragalus membranaceus (Fisch.) Bge.; Rhizoma Dioscoreae, Chinese yam, is the rhizome from the plant Dioscorea opposite Thunb.; Radix Paeoniae Alba, white peony root, is the root without bark of the plant Paeonia lactiflora Pall.; Herba Ecliptae, false daisy. is the arial part of the plant Eclipta prostrata L.; Fructus Mori, mulberry, is the fruit of the plant Morus alba L.; Radix Curcumae, curcuma tuber, is the root and rhizome of the plant Curcuma wenyujin Y. H. Chen et C. Ling, Curcuma kwangsiensis S. Lee et C. F. Liang, Curcuma longa L., Curcuma phaeocaulis Val.; Herba Cynomorii, cynomorium, is the fleshy stem of the plant Cynomorium songaricum Rupr.

One objective of the present invention is to provide an herbal composition that promotes fertility in both males and females.

Another objective is to provide a plurality of herbal ingredients that are healthy for the kidney, liver, and spleen.

Yet another objective is to provide an herbal composition that consists of easily available herbal ingredients.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a table of an exemplary herbal composition for promoting fertility in males and females, showing various ranges of weight percentage for the herbal ingredients, in accordance with an embodiment of the present invention.

Like reference numerals refer to like parts throughout the various views of the drawings.

### DETAILED DESCRIPTION OF THE INVENTION

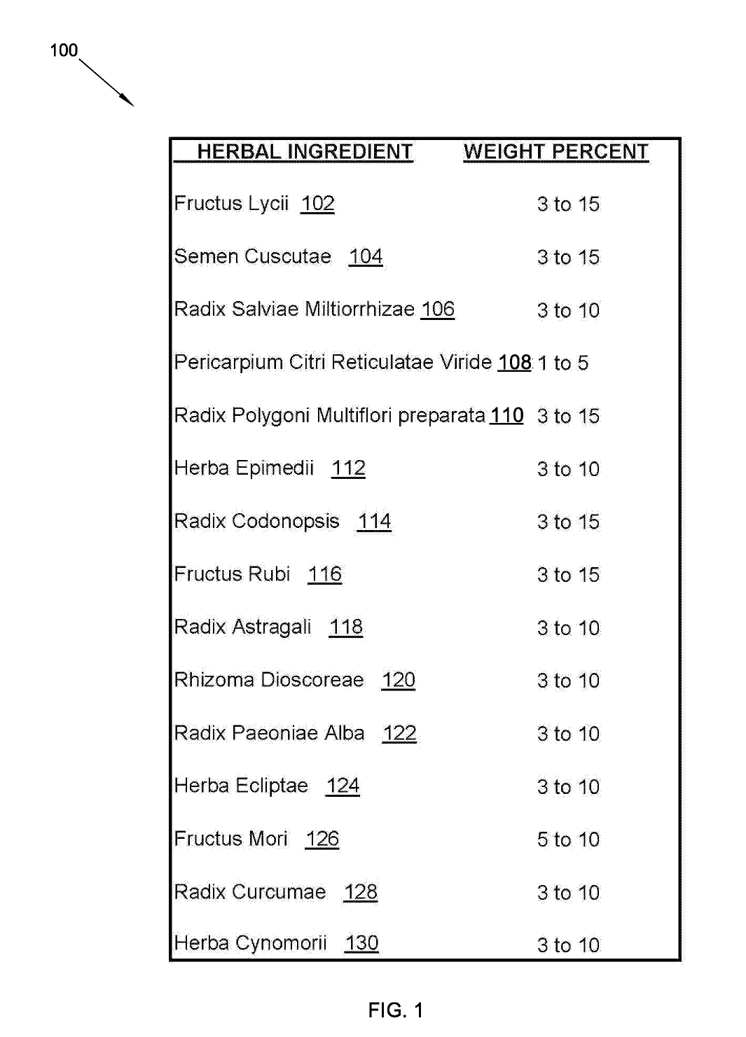

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

An herbal composition 100 that enhances and promotes fertility in both males and females is referenced in the table of FIG. 1. The herbal composition 100 consists of a plurality of herbal ingredients that combine to create a synergy that enhances fertility by nourishing the kidney, improving circulation of Qi and blood, and balancing the forces of Yin and Yang. The herbal composition 100 consists of a laundry list of herbal ingredients known in traditional Chinese medicine to address fertility issues. The present disclosure provides a novel mixture of the ingredients, including unique processing, extraction, and purification methods for optimizing efficacy of the herbal ingredients.

The individual herbal ingredients are efficacious for nourishing the kidney, regulating the liver and spleen. The kidney is the main focus in traditional Chinese medicine treatment of both female and male infertility. Those skilled in the art will recognize that traditional Chinese medicine teaches that the kidney is the congenital base of life. The kidney in traditional Chinese medicine is considered as a functional unit of controlling reproduction and growth. Modern research has demonstrated that the kidney functions cover the regulation of core physiological axis of hypothalamus-pituitary, including the hypothalamic-pituitary-gonadal axis (HPG axis).

Furthermore, functions of liver and spleen in traditional Chinese medicine also impact male and female fertility. Traditional Chinese medicine teaches that aging starts with decline of kidney function. Thus, strengthening the function of the kidney is one of the important strategies of anti-aging and treatment for infertility in traditional Chinese medicine.

The herbal composition 100 also works to improve circulation of Qi and blood. It is known that, poor blood circulation and imbalance of Yin and Yang also affect the sexual function and fertility of both male and female. The herbal composition 100 is also effective for both enhancing blood circulation and balancing the Yin and Yang. Thus, the opposite or contrary forces that may be inhibiting fertility are made more complementary, interconnected, and interdependent through consumption of the herbal composition 100. Furthermore, the herbal ingredients, when mixed together, create activities of anti-inflammation, anti-oxidation, anti-pathogenic microorganisms, and are able to improve microcirculation. This results in enhanced gonadal function to improve fertility.

The composition 100 consists of the following herbal ingredients mixed in varying weight percentages: Fructus Lycii 102 (or goji berry); Semen Cuscutae 104 (or dodder seed); Radix Salviae Miltiorrhizae 106 (or red sage root); Pericarpium Citri Reticulatae Viride preparata 108 (or green orange peel); Radix Polygoni Multiflori preparata 110 (or fleece flower root); Herba Epimedii 112 (or horny goat weed); Radix Codonopsis 114 (or codonopsis); Fructus Rubi 116 (or raspberry); Radix Astragali 118 (or astragalus); Rhizoma Dioscoreae 120 (or Chinese yam); Radix Paeoniae Alba 122 (or white peony root); Herba Ecliptae 124 (or false daisy); Fructus Mori 126 (or mulberry); Radix Curcumae 128 (or curcuma tuber); and Herba Cynomorii 130 (or cynomorium). The ingredients may be mechanically processed prior to mixing. The mixing process is at least partially dependent on the desired means of administering the composition 100.

The weight percentage (w %) for each herbal ingredient is the ratio of one herbal ingredient with mass (m) to the mass of the total mixture (mₜ) of herbal ingredients. The formula used in the present invention is as follows:

w %=m/mₜ

As the table in FIG. 1 references, there are various embodiments of weight percentages for the ingredients of the herbal composition 100. One possible range of weight percentages for the herbal ingredients may include: about 3 to 15 weight % Fructus Lycii 102, about 3 to 15 weight % Semen Cuscutae 104, about 3 to 10 weight % Radix Salviae Miltiorrhizae 106, about 1 to 5 weight % Pericarpium Citri Reticulatae Viride preparata 108, about 3 to 15 weight % Radix Polygoni Multiflori preparata 110, about 3 to 10 weight % Herba Epimedii 112, about 3 to 15 weight % Radix Codonopsis 114, about 3 to 15 weight % Fructus Rubi 116, about 3 to 10 weight % Radix Astragali 118, about 3 to 10 weight % Rhizoma Dioscoreae 120, about 3 to 10 weight % Radix Paeoniae Alba 122, about 3 to 10 weight % Herba Ecliptae 124, about 5 to 10 weight % Fructus Mori 126, about 3 to 10 weight % Radix Curcumae 128, and about 3 to 10 weight % Herba Cynomorii 130.

Another alternative embodiment of the herbal composition 100 references yet a more specific weight percentage for each herbal ingredient, as follows: Fructus Lycii 8 weight %, Semen Cuscutae 15 weight %, Radix Salviae Miltiorrhizae 10 weight %, Pericarpium Citri Reticulatae Viride preparata 108 1 weight %, Radix Polygoni Multiflori preparata 15 weight %, Herba Epimedii 3 weight %, Radix Codonopsis 3 weight %, Fructus Rubi 3 weight %, Radix Astragali 118 root 10 weight %, Rhizoma Dioscoreae 8 weight %, Radix Paeoniae Alba 3 weight %, Herba Ecliptae 7 weight %, Fructus Mori 8 weight %, Radix Curcumae 3 weight %, and Herba Cynomorii 3 weight %.

Another alternative embodiment of the herbal composition 100 may include, the following herbal ingredients: Fructus Lycii 15 weight %, Semen Cuscutae 3 weight %, Radix Salviae Miltiorrhizae 3 weight %, Pericarpium Citri Reticulatae Viride preparata 5 weight %, Radix Polygoni Multiflori preparata 3 weight %, Herba Epimedii 10 weight %, Radix Codonopsis 5 weight %, Fructus Rubi 15 weight %, Radix Astragali 3 weight %, Rhizoma Dioscoreae 3 weight %, Radix Paeoniae Alba 10 weight %, Herba Ecliptae 10 weight %, Fructus Mori 5 weight %, Radix Curcumae 5 weight %, and Herba Cynomorii 5 weight %.

Another alternative embodiment of the herbal composition 100 may include, the following: Fructus Lycii 3 weight %, Semen Cuscutae 5 weight %, Radix Salviae Miltiorrhizae 5 weight %, Pericarpium Citri Reticulatae Viride preparata 3 weight %, Radix Polygoni Multiflori preparata 5 weight %, Herba Epimedii 5 weight %, Radix Codonopsis 15 weight %, Fructus Rubi 6 weight %, Radix Astragali 5 weight %, Rhizoma Dioscoreae 10 weight %, Radix Paeoniae Alba 5 weight %, Herba Ecliptae 3 weight %, Fructus Mori 10 weight %, Radix Curcumae 10 weight %, and Herba Cynomorii 10 weight %.

Another alternative embodiment of the herbal composition 100 may include, the following: Fructus Lycii 5 weight %, Semen Cuscutae 10 weight %, Radix Salviae Miltiorrhizae 7 weight %, Pericarpium Citri Reticulatae Viride preparata 2 weight %, Radix Polygoni Multiflori preparata 8 weight %, Herba Epimedii 7 weight %, Radix Codonopsis 7 weight %, Fructus Rubi 9 weight %, Radix Astragali 7 weight %, Rhizoma Dioscoreae 5 weight %, Radix Paeoniae Alba 7 weight %, Herba Ecliptae 5 weight %, Fructus Mori 6 weight %, Radix Curcumae 8 weight %, and Herba Cynomorii 7 weight %.

As discussed above, the individual herbal ingredients that make up the herbal composition 100 create a synergy in which the whole is greater than the individual ingredients for enhancing fertility in males and females.

As used in the present disclosure, one of the herbal ingredients is Lycium Fruit 102, known as goji berry or wolfberry in English and Gou Qi Zi in mandarin, is the fruit of the plant Lycium barbarum, Lycium Fruit has been used in traditional Chinese medicine (TCM) for more than 2000 years. The polysaccharides in Lycium Fruit 102 have been shown in studies to improve general well-being and immune functions, have anti-oxidative, anti-aging and antitumor activities, exhibit significant hypoglycemic and cardioprotective effects as well as antiviral and anti-inflammatory activities, protect the liver and nerves from injury. Research also revealed that the polysaccharides exhibit the ability of protecting the testis from toxic insults, thus, are beneficial to male reproduction by increasing the quality, quantity, and motility of sperm, improving sexual performance.

Another herbal ingredient is Semen Cuscutae 104, which is the seed of the plant Cuscuta chinensis Lam. or Cuscuta japonica Choisy. Semen Cuscutae 104, known as cuscuta seed in English and Tu Si Zi in mandarin, has long been considered a fabulous mild tonic used in Chinese herbal remedies. These two species are plants in the family Convolvulaceae. When used as one of medicinal herbs, the aboveground part should be reaped and dried in autumn when the fruits are ripe. And the seeds are thus collected. Medicinally the dodder seed 104 is used raw or cooked and mashed.

Yet another herbal ingredient used in the herbal composition 100 is Radix Salviae Miltiorrhizae 106, known as red sage root, Chinese sage in English and Dan Shen in mandarin. Radix Salviae Miltiorrhizae 106 is the root and rhizome of the plant Salvia miltiorrhiza Bge,. Radix Salviae Miltiorrhizae 106 has been widely used in China and, to a lesser extent, in Japan, the United States, and European countries for the treatment of cardiovascular and cerebrovascular diseases. In China, Radix Salviae Miltiorrhizae (alone or combined with other Chinese herb medicine) was applied to the treatment of variety of diseases such as angina pectoris, myocardial infarction, hypertension, hyperlipidemia, and acute ischemic stroke.

Yet another herbal ingredient used in the herbal composition 100 is Pericarpium Citri Reticulatae Viride preparata 108 is the rind of immature green fruit of the plant Citrus reticulate Blanco that is processed with vinegar. Pericarpium Citri Reticulatae Viride preparata 108 is known as processed unripe tangerine peel or processed immature mandarin orange peel in English and Cu Zhi Qing Pi in mandarin.

The herbal composition 100 also consists of Radix Polygoni Multiflori preparata 110, known as the processed fleece flower root or Chinese knotweed in English and Zhi He Shou Wu in mandarin, is the processed root of the plant Polygonum multiflorum Thunb. Radix Polygoni Multiflori preparata 110 is used to restore blood, liver and kidneys and to increase mental clarity. It is often prescribed for insomnia, dizziness, prematurely gray hair caused by blood deficiency.

Yet another herbal ingredient used in the herbal composition 100 is Herba Epimedii 112, the anal part of the plant Epimedium brevicornum Maxim. Herba Epimedii 112 is known as epimedium, barrenwort, bishop's hat, fairy wings in English and Yin Yang Huo in mandarin. Herba Epimedii 112 contains icariin, which has demonstrated in vitro PDE5 inhibitory properties. Icariin is purported to work by increasing levels of nitric oxide, which relax smooth muscle. It has been demonstrated to relax rabbit penile tissue by nitric oxide and PDE5 activity. Other research has demonstrated that injections of Epimedium extract directly into the penis of the rat results in an increase in penile blood pressure.

Another herbal ingredient is Radix Codonopsis 114, known as codonopsis root and pilose Asia bell root in English and Dang Shen in mandarin, is the root of the plant Codonopsis pilosula (Franch) Nannf., Codonopsis pilosula Nannf. var. moderta (Nannf.) L., or Codonopsis tangshen Oliv. Radix Codonopsis 114 is used to treat HIV infection and to protect cancer patients against side effects of radiation treatment. It is also to boost the immune system; and to treat weakness, loss of appetite (anorexia), chronic diarrhea, shortness of breath, noticeable heartbeat (palpitations), asthma, cough, thirst, and diabetes.

Another herbal ingredient is Fructus Rubi 116, known as raspberry, palm-leaf raspberry fruit or rubus in English and Fu Pen Zi in mandarin, is the fruit of the plant Rubus chingii Hu. Pharmacological studies showed that a water extract of Fructus Rubi 116 can act directly on rat Leydig cells (cells in the tests that secrete the hormone testosterone) to promote the activity of steroid synthesizing enzymes and to inhibit their degradation. As a consequence, more testosterone was synthesized and blood testosterone levels were elevated in the rats, and it also has estrogenic effect.

Another herbal ingredient is Radix Astragali 118, known as astragalus, mongolian milkvetch root or membranous milkvetch root in English and Huang Qi in mandarin, is the root of the plant Astragalus membranaceus Bge. Radix Astragali 118 has been used in traditional Chinese medicine for centuries. Its main use has been to boost the body's immune system. But it also has been used to treat other conditions, including heart disease. It has antioxidant effects that inhibit free radical production.

Another herbal ingredient utilized in the composition 100 is Rhizoma Dioscoreae 120, known as Chinese yam, dioscorea or common yam rhizome in English and Shan Yao in mandarin, is the rhizome of the plant Dioscorea opposite Thunb. Rhizoma Dioscoreae 120 is claimed to contain human hormones and promoted as a medicine for a variety of purposes, including cancer prevention and the treatment of Crohn's disease and whooping cough.

Yet another herbal ingredient used in the herbal composition 100 is the Radix Paeoniae Alba 122, known as white peony root in English and Bai Shao in mandarin, is the root without bark of the plant Paeonia lactiflora Pall. Radix Paeoniae Alba 122 is used for menstrual cramps, polycystic ovary syndrome, premenstrual syndrome (PMS) in women, and also used for viral hepatitis, liver cirrhosis, upset stomach, muscle cramps.

Another herbal ingredient is Herba Ecliptae 124, known as false daisy or eclipta in English and Mo Han Lian or Han Lian Cao in mandarin, is the arial part of the plant Eclipta prostrate L. Pharmacological studies showed that Herba Ecliptae 124 can protect liver and regulate immune function. It also has hemostasis, hypoglycemic, anti-tumor, anti-inflammatory, anti-anoxia and anti-venom effects.

Another herbal ingredient is Fructus Mori 126, known as mulberry, mulberry fruit in English and Sang Shen in mandarin, is the fruit of the plant Morus alba L. Pharmacological studies showed that Fructus Mori has antioxidant, hypoglycemic, lipid-lowering activities and anti-cancer mutation effects. It can also regulate the immune function.

Another herbal ingredient is the Radix Curcumae 128, known as curcuma tuber, wild turmeric, curcuma in English and Yu Jin or Wen Yu Jin in mandarin, is the root and rhizome of the plant Curcuma wenyujin Y. H. Chen et C. Ling, Curcuma kwangsiensis S. Lee et C. F. Liang, Curcuma longa L., or Curcuma phaeocaulis Val. The curcuma tuber 128 has been revered as a food and medicine for thousands of years. Having a long history of use for its anti-inflammatory effects, the curcuma tuber 128 (or turmeric) is an herb of major importance in the East and has been used extensively in the Indian and Chinese systems of medicine. The curcuma tuber 128 has been investigated for its anti-inflammatory, antibacterial, antiparasitic, choleretic, antiviral, antiplatelet, analgesic, antioxidant, anticancer, antihepatotoxic and antitumor effects.

Another herbal ingredient is Herba Cynomorii 130, known as cynomorium in English and Suo Yang in mandarin, is the fleshy stem of the plant Cynomorium songaricum Rupr., The primary functions of Herba Cynomorii in traditional Chinese medicine are to tonify the kidney, fortify yang, nourish the blood and strengthen the sinews. It is used to treat impotence in men, and infertility and lack of libido in women. In both genders, it is used for strengthening the back, legs and skeleton, and weakness of the tendons due to kidney yang deficiency.

In some embodiments, the herbal composition 100 may be administered in at least one of the following ways: a pill, a dripping pill, a tablet, an orally disintegrating tablet, a granule, a powder, a lozenge, a capsule, a cream, a suppository, and an oral liquid.

The herbal composition 100 consists of a plurality of herbal ingredients prepared through processing methods, extraction methods, and purification methods. Those skilled in the art will recognize that each herbal ingredient may be processed, extracted, and purified differently. In some embodiments, the processing methods include at least one member selected from the group consisting of: washing, steaming, extracting, roasting, herb frying, salt frying, honey frying, wine frying, earth frying, vinegar frying, calcining drying, heating, and grinding.

In some embodiments, the extraction methods include at least one member selected from the group consisting of: soaking, heating, steaming, evaporation, compressing, and solvent extraction with at least one of the following: water, alcoholic solvents mixed with water, petroleum ether, hexane, diethyl amine, diethyl ether, cyclohexane, tert-butyl alcohol, isopropanol, Acetonitrile, aceton, ethanol, methyl isobutyl ketone, isobutyl alcohol, 1-propanol, methyl ethyl ketone, 2-butanol, isoamyl alcohol, 1-butanol, diethyl ketone, 1-octanol, p-xylene, m-xylene, toluene, dimethoxyethane, benzene, butyl acetate, 1-chlorobutane, tetrahydrofuran, ethyl acetate, o-xylene, hexamethylphosphorus triamide, 2-ethoxyethyl ether. N,N-dimethylacetamide, diethylene glycol dimethyl ether, N,N-dimethylformamide, 2-methoxyethanol, pyridine, propanoic acid, 2-methoxyethyl acetate, benzonitrile, 1-Methyl-2-pyrrolidinone, hexamethylphosphoramide, 1,4-dioxane, acetic acid, acetic anhydride, dimethyl sulfoxide, chlorobenzene, ethylene glycol, diethylene glycol, propylene carbonate, formic acid, 1,2-dichloroethane, glycerin, carbon disulfide, 1,2-dichlorobenzene, methylene chloride, nitromethane, 2,2,2-trifluoroethanol, chloroform, 1,1,2-trichlorotrifluoroethane, carbon tetrachloride, and tetrachloroethylene.

In some embodiments, the purification methods include at least one member selected from the group consisting of: column separation, solvent partition, evaporation, and solvent precipitation.

The safety of the composition 100 was tested in laboratories with mice and standard scientific procedure known in the art. An acute toxicity experiment and a sub-chronic toxicity experiment showed negligible amounts of toxicity, indicating the composition 100 is safe for human consumption.

Specifically, acute and chronic toxicities were evaluated for the herbal composition 100 using specific-pathogen-free (SPF) Sprague-Dawley (SD) rats. The toxicological evaluation included clinical observation for signs of toxicity, mortality, body weight, food consumption, hematology, blood biochemistry, urinalysis, organ weights, and gross and microscopic pathological examination of organs and tissues.

In one experiment, a study for acute toxicity was performed on SD rats of both sexes at a single dose by oral gavage. The rats were randomly divided into two groups, 20 rats in each (half male and half female). Group 1 served as the control group received distilled water in a volume of 2.5 ml/100 g body weight, whereas group 2 was the treatment group that received composition 100-distilled water suspension at the dose of 20 g/kg body weight. Following the dosing, the rats were observed daily and weighed on day 1, 4, 7, 11 and 14. A gross necropsy examination was performed on all rats at the time of scheduled euthanasia (day 14).

No mortality occurred during the study. The body weight of rats in both control and treatment groups was gradually increased with the time during the experimental periods and there was no significant difference between the groups. No notable clinical abnormalities, such as rough coat, decreased activity, congested breathing, dark material around the facial area, decreased defecation, salivation, soft stools and urine/fecal stain, were observed during the study. Neither found abnormal pathological change of all internal organs at necropsy in both groups. The conclusion was that under the conditions of this study, the maximum tolerable dose (MTD) of the formula to SD rats at single dose given by oral gavage was greater than 20 g/kg.

In another experiment embodiment, a study for chronic toxicity was performed to evaluate chronic toxic effects of the herbal composition 100. The toxicological evaluations included clinical observation for signs of toxicity, mortality, body weight, food consumption, urinalysis, hematology, serum biochemistry, and organ weights, gross and microscopic pathology.

The study was performed using healthy male and female SD rats at 7-9 weeks of age. The rats were randomly divided into 4 groups. 15 males and 15 females were assigned to each group. The rats were administered the test substances for a period of 90 days by oral gavage. Group I served as the vehicle-control group received only distilled water (2.0 ml/day/100 g body weight), whereas the rats in group II, III and IV were respectively given the formula suspension at the dose of 2000, 4000 and 8000 mg/day/kg body weight.

At the 90th day of the administration, 20 rats in each group were sacrificed. The remaining 10 in each group were sacrificed 30 days later. The indicators for detection include: general condition, weights of the body and organs; food consumption, hematology, serum biochemistry, electrolyte, coagulation index, sex hormones, urinalysis, gross anatomy, and histopathology.

All rats were observed at home cage once a day for general health condition, signs of toxicity, morbidity, and mortality prior to initiation of the treatment and then once per day during the treatment and recovery period. Findings were recorded using a scoring system. Any deviations from normal will be recorded in terms of nature and severity, date and time of onset, duration and progress of the observed response. The visual observation include changes in skin, fur, eyes, mucous membranes, occurrence of secretions and excretions and autonomic activity such as lacrimation, piloerection, pupil size, and unusual respiratory patterns as well as activity level and change in behavior. Furthermore, potential changes in gait, posture and response to handling as well as the presence of somnolence, trembling, clonic or tonic movements, stereotypes or bizarre behavior were to be recorded. Any rat that died during the test period was examined pathologically.

Body weights of all rats were recorded right before initiation of the treatment, then once a week during the treatment period, and also at the end of day 90 and day 120. Group mean body weight was calculated at each measurement for each sex. In addition, final body weights of all rats at terminal sacrifice after overnight fasting were recorded and used for calculation of relative organ weights.

Food consumption for each cage was measured once before initiation of treatment and once weekly during the treatment period. The obtained value was divided by the number of rats alive in the cage and by the number of days for measurement to obtain mean daily food consumption per rat in the cage. Group mean food consumption (g/rat/day) for each sex was calculated from these individual cage values at each week. The overall average group mean food consumption throughout the treatment period was also calculated for each sex using mean weekly data.

Hematological examinations were performed on all rats at the end of day 90 of treatment or day 120 of recovery. All surviving rats were fasted overnight but with free access of water and afterwards were anesthetized by intraperitoneal injection with 2.0% pentobarbital sodium (40 mg/kg for females and 50 mg/kg for males) for blood collection from a common carotid artery. A part of the sample was poured into a cup containing ethylenediaminetetraacetic acid (EDTA) Blood smears were prepared for these rats using the blood samples described above. The smears were stained with brilliant cresyl blue for reticulocytes count.

The following parameters were measured: red blood cell (RBC), hemoglobin concentration (HGB), hematocrit (HCT), mean corpuscular volume (MCV), mean hemoglobin (MCH), mean corpuscular hemoglobin concentration (MCHC), red cell distribution width—coefficient of variation (RDW-CV), white blood cell (WBC) , neutrophils (NEUT), lymphocytes (LYM), monocytes (MON), osinophils (EOS), basophils (BAS), platelet count (PLT), mean platelet volume (MPV), mean platelet distribution (PDW), reticulocyte count (Reti).

Blood biochemical examinations were performed on all rats at the end of 90-day treatment and end of the 30-day recovery. Serum samples obtained from the blood mentioned in the above section on Hematology were used. The following parameters were determined: alkaline phosphatase (ALP/GPT), aspartate aminotransferase (AST/GOT), alkaline phosphatase (ALP), total protein (TP), albumin (ALB), globulin (GLB), albumin/globulin (A/G), total bilirubin (TBIL), creatinine (CREA), urea (UREA), uric acid (UA), creatine kinase (CK), triglyceride (TG), total cholesterol (CHOL), lactate dehydrogenase (LDH), glucose (GLU), calcium (Ca), blood urea nitrogen (BUN).

The analysis of the hormones follicle stimulating hormone (FSH), luteinizing hormone (LH), estradiol (E2) and testosterone (T) were carried out using enzyme-linked immunoabsorbent assay (ELISA) in serum samples.

Serum sodium (Na), potassium (K), and chloride (Cl) were measured for serum electrolytes.

Prothrombin time (PT) and activated partial thromboplastin time (APTT) were determined on plasma samples obtained by centrifugation of the mixture with nine parts of the native blood and one part of 3.2% sodium citrate solution using an automated blood coagulation analyzer.

Urinalysis was performed on all rats at the end of day 90 of treatment or day 120 of recovery. The following parameters were determined on fresh urine samples: specific gravity, pH, protein, glucose, V-C, ketones, bilirubin, occult blood, and urobilinogen. Appearance, volume and sediments were determined on the urine sample pooled overnight from each rat in a metabolic cage.

At the end of 90-day treatment and the 30-day recovery period, internal organs of the rats were weighted and examined. These include the brain, spine, pituitary gland, eyes, thymus, heart, aorta, lungs, liver, spleen, pancreas, kidneys, adrenal gland, adrenal gland, trachea, esophagus, stomach, duodenum, jejunum, ileum, appendix, colon, testicular, seminal vesicle, prostate, ovaries, fallopian tubes, uterus, cervix, vagina, thyroid (including parathyroid), salivary gland, Harderian gland, sciatic nerve, breast, bone marrow (sternum), bone (femur), lymph nodes, skeletal muscle, skin.

During the experiment, two rats died in pill treatment groups. The histopathology exam reveals that one died from perfusion of the formula suspension into the lungs caused by operation mistake. Another remains undetermined. Observed by the microscope, no significant pathological changes of the organs were found, but examination showed the death has no correlation with the formula.

Intermittent convulsions were observed on two rats in the 2000 and 4000 mg/day/kg Qilin Pill treatment groups, but recovered later. Besides these two, no obvious abnormality was observed in all groups in general conditions. In another word, the behavior and appearance of the rats, and the stool color and shape were all normal.

During the period of the study, the body weight of male and female rats in each group increased continuously. There were no significant differences in body weight in the three Qilin Pill treatment groups compared with the control group. Except on day 113 and 120 when the body weight of male rats in 4000 mg/day/kg Qilin Pill treatment group was significantly lower that that in the control group (P<0.05), the body weight of the male rats in the 3 treatment groups had no significant differences compared with the control group.

These results suggest that the decrease in the body weight on this only two days observed in these male rats might not be considered related to the test substances, but rather a result of chance on biological variation. In addition, if this decrease in the body weight relates to any toxicity, it will be only a reversible one.

Regarding food intake, there was no difference between the control group and the three treatment groups.

As for the effect on hematological parameters, after 90 days of treatment, male rats in the group treated with 8000 mg/day/kg composition 100 pill showed significant decrease in hemoglobin (HGB) content (P<0.01) compared with the control group. The changes returned to normal at the end of the 30-day recovery period, indicating that the change was transient.

There were no significant differences in other hematological parameters of both male and female rats that treated with 8000 mg/day/kg pill compared with the control group. And no other hematological parameters in the three pill treatment groups was found compared with the control group. At the end of the 30-day recovery period, no significant differences of hematological parameters treated between the three pill groups and the control group.

As for the effect on serum biochemical and sexual hormone parameters, after 90 days of treatment, no significant differences of the biochemical in females were found between the pill treatment groups and the control group. But to the male rats, albumin and globulin ratio (A/G) decreased significantly (P<0.01) in the group treated with 2000 mg/day/kg pill, meanwhile, total bilirubin (TBIL) significantly increased (P<0.05) in the group treated with 4000 and 8000 mg/day/kg pill in comparison with the control group. No significant difference of the other serum biochemical parameters in male rats was found between the three pill treatment groups and the control group.

About hormones, luteinizing hormone (LH) of the female rats in the 2000 mg/day/kg and 8000 mg/day/kg pill treatment groups showed significant decreases (P<0.01) compared with the control group after 90 days of treatment. Meanwhile, follicle-stimulating hormone (FSH) of the male rats treated with 2000 mg/day/kg pill showed significant increases (P<0.05) in comparison with the control group.

At the end of the 30-day recovery period, the level of luteinizing hormone (LH) of the female rats in 2000 mg/day/kg and 4000 mg/day/kg treated group significantly decreased (P<0.01). No significant difference of the hormone parameters was found in male rats treated with three pill doses compared with the control group.

These significant changes of the above serum biochemical and sexual hormone parameters had no dose-effect relationship, thus, were considered as a physiological variation, no toxicological meaning.

As for the effect on serum electrolytes, after 90 days of treatment, except serum potassium (K) of the female rats in the 2000 mg/day/kg group showed significant increases (P<0.05) compared with the control group, there was no significant difference of serum electrolytes between the three pill groups and the control group. Neither at the end of the 30-day recovery period.

As for the effect on the coagulation index, after 90 days of treatment, except plasma prothrombin time (APTT) of the male rats in the 8000 mg/day/kg group showed significant reduced compared with the control group (P<0.05), there were no significant differences in coagulation index among both male and female rats in the three pill treatment groups and the control group.

At the end of the 30-day recovery period, no significant differences of all coagulation indexes among both male and female rats treated with the three doses of pill doses and the control group.

As for the effect on the urinalysis parameters, in the 90-day of treatment period and 30-day of recovery period, the appearance of urine was clear and in pale yellow color in all three pill groups. There was no significant difference in all urinalysis parameters among the three pill doses and the control group.

As for the effect on the organ weight and organ coefficient, at the end of 90-day treatment and 30-day recovery period, there were no significant differences in organ weight and organ coefficient of female rats in the three pill treatment groups compared with the control group.

After 90 days of treatment, except weight of the male testicular of the 2000 mg/day/kg group showed significant reduced compared with the control group (P<0.05), there was no significant difference of the other organ weight and organ coefficient of male rats among the three pill groups compared to the control group.

At the end of the 30-day recovery period, spleen weight of the male rats in the 4000 mg/day/kg and 8000 mg/day/kg groups showed significant decrease compared with the control group (P<0.05). And spleen coefficient of the male rats in the 8000 mg/day/kg group showed significant decrease compared with the control group (P<0.01).

During the experiment, a histopathology examination of the rats was performed. During the experiment on day 42 after administration, one male rat in the 2000 mg/day/kg died and the histopathological finding reveals that the brow settlings in stomach, bilateral bronchiectasis, terminal bronchioles and alveolus and the brow settlings may be residue of the pill. Congestion caused by sudden death was observed in liver, spleen, kidneys and adrenal glands and no significant fatal pathological changes were found in other organs of the rat. The rat died from perfusion of pill-distilled water suspension into the lungs due to the operation mistake.

During the experiment on day 80 after administration, one male rat in the 4000 mg/day/kg died and observed by the microscope, no significant fatal pathological changes were found in every organ of the rat. The cause of death of the rat No. 62 remains undetermined.

As a result, no significant composition 100 pill-caused pathological change was found in the three treatment groups when compared to the control group at the end of 90-day treatment. And no significant delayed and accumulative pathological toxicity changes were observed in the three treatment groups when compared with the control group either at the end of the 30-day recover period.

In conclusion of the experiment, it was found that toxicology of the pill form of the herbal composition 100 was evaluated using SD rats. The evaluation included clinical observation for signs of toxicity, mortality, body weight, food consumption, urinalysis, hematology, serum biochemistry, sex hormones, electrolyte, coagulation index, urinalysis, organ weights, gross and microscopic histopathology.

Under the conditions of this study, no significant dosage-toxicity related pathological change was observes in both male and female rats between pill treated groups and the control group. Neither at the end of the 30-day recovery period. The result suggests that no-observed-adverse-effect level (NOAEL) of the formula is above 8000 mg/kg, which is at least 22 times higher than the dosage used in clinic for patients (the pill form of the composition 100 is administrated at 12-18 g/day in clinic, if calculated based on a patient with 50 kg body weight, it is 360 mg/kg of body weight).

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. An herbal composition for enhancing fertility in males and females, the composition comprising:
about 3 to 15 weight percent Fructus Lycii, about 3 to 15 weight percent Semen Cuscutae, about 3 to 10 weight percent Radix Salviae Miltiorrhizae, about 1 to 5 weight percent Pericarpium Citri Reticulatae Viride preparata, about 3 to 15 weight percent Radix Polygoni Multiflori preparata, about 3 to 10 weight percent Herba Epimedii, about 3 to 15 weight percent Radix Codonopsis, about 3 to 15 weight percent Fructus Rubi, about 3 to 10 weight percent Radix Astragali, about 3 to 10 weight percent Rhizoma Dioscoreae, about 3 to 10 weight percent Radix Paeoniae Alba, about 3 to 10 weight percent Herba Ecliptae, about 5 to 10 weight percent Fructus Mori, about 3 to 10 weight percent Radix Curcumae, and about 3 to 10 weight percent Herba Cynomorii.

2. The composition of claim 1, wherein the composition is administered in at least one of the following ways: oral, topical, suppository, intravenous, intradermic, intragaster, intramuscular, and intraperitoneal.

3. The composition of claim 1, wherein the composition includes at least one member selected from the group consisting of: a tablet, an orally disintegrating tablet, a pill, a dripping pill, a lozenge, a capsule, a cream, a granule, a suppository, and an oral liquid.

4. The composition of claim 1, wherein the composition is administered in a dosage ranging from 0.01 to 500 miligrams per kilogram of body weight.

5. The composition of claim 1, wherein the composition consists of a plurality of herbal ingredients prepared through processing methods, extraction methods, and purification methods.

6. The composition of claim 5, wherein the processing methods include at least one member selected from the group consisting of: washing, steaming, extracting, roasting, herb frying, salt frying, honey frying, wine frying, earth frying, vinegar frying, calcining drying, heating, and grinding.

7. The composition of claim 6, wherein the extraction methods include at least one member selected from the group consisting of: soaking, heating, steaming, evaporation, compressing, and solvent extraction with at least one of the following: water, alcoholic solvents mixed with water, petroleum ether, hexane, diethyl amine, diethyl ether, cyclohexane, tert-butyl alcohol, isopropanol, acetonitrile, aceton, ethanol, methyl isobutyl ketone, isobutyl alcohol, 1-propanol, methyl ethyl ketone, 2-butanol, isoamyl alcohol, 1-butanol, diethyl ketone, 1-octanol, p-xylene, m-xylene, toluene, dimethoxyethane, benzene, butyl acetate, 1-chlorobutane, tetrahydrofuran, ethyl acetate, o-xylene, hexamethylphosphorus triamide, 2-ethoxyethyl ether. N,N-dimethylacetamide, diethylene glycol dimethyl ether, N,N-dimethylformamide, 2-methoxyethanol, pyridine, propanoic acid, 2-methoxyethyl acetate, benzonitrile, 1-Methyl-2-pyrrolidinone, hexamethylphosphoramide, 1,4-dioxane, acetic acid, acetic anhydride, dimethyl sulfoxide, chlorobenzene, ethylene glycol, diethylene glycol, propylene carbonate, formic acid, 1,2-dichloroethane, glycerin, carbon disulfide, 1,2-dichlorobenzene, methylene chloride, nitromethane, 2,2,2-trifluoroethanol, chloroform, 1,1,2-trichlorotrifluoroethane, carbon tetrachloride, and tetrachloroethylene.

8. The composition of claim 7, wherein the purification methods include at least one member selected from the group consisting of: column separation, solvent partition, evaporation, and solvent precipitation.

9. The composition of claim 1, wherein Fructus Lycii, the goji berry, is the fruit of the plant Lycium barbarum L.; Semen Cuscutae , the dodder seed, is the seed of the plant Cuscuta chinensis Lam.; Radix Salviae Miltiorrhizae, the red sage root, is the root and rhizome of the plant Salvia miltiorrhiza Bge.; Pericarpium Citri Reticulatae Viride preparata, the processed green orange peel, unripe tangerine peel, an immature mandarin orange peel, is the rind of green fruit of the plant Citrus reticulate Blanco; Radix Polygoni Multiflori preparata, the processed fleece flower root or Chinese knotweed, is the processed root of the plant Polygonum multiflorum Thunb.; Herba Epimedii, the horny goat weed, is the arial part of the plant Epimedium brevicornum Maxim., Epimedium sagittatum (Sieb. Et Zucc.) Maxim., Epimedium pubescens Maxim., Epimedium wushanense T. S. Ying, Epimedium koreanum Nakai; Radix Codonopsis, the codonopsis root, is the root of the plant Codonopsis pilosula (Franch) Nannf., Codonopsis pilosula Nannf. var. moderta (Nannf.) L., Codonopsis tangshen Oliv.; Fructus Rubi, the raspberry, is the fruit of the plant Rubus chingii Hu; Radix Astragali, the astragalus root is the root of the plant Astragalus membranaceus (Fisch.) Bge. var. mongholicus (Bge.) Hsiao, Astragalus membranaceus (Fisch.) Bge.; Rhizoma Dioscoreae, the Chinese yam, is the rhizome from plant Dioscorea opposite Thunb.; Radix Paeoniae Alba, the white peony root, is the root without bark of the plant Paeonia lactiflora Pall.; Herba Ecliptae, the false daisy, is the arial part of the plant Eclipta prostrata L.; Fructus Mori, the mulberry, is the fruit of the plant Morus alba L.; Radix Curcumae, the curcuma tuber, is the root and rhizome of the plant Curcuma wenyujin Y. H. Chen et C. Ling, Curcuma kwangsiensis S. Lee et C. F. Liang, Curcuma longa L., Curcuma phaeocaulis Val; Herba Cynomorii, the cynomorium is the fleshy stem of the plant Cynomorium songaricum Rupr.

10. An herbal composition for enhancing fertility in males and females, the composition consisting of:
About 8 weight percent Fructus Lycii, about 15 weight percent Semen Cuscutae, about about 10 weight percent Radix Salviae Miltiorrhizae, about 1 weight percent, Pericarpium Citri Reticulatae Viride preparata, about 15 weight percent Radix Polygoni Multiflori preparata, about 3 weight percent Herba Epimedii, about 3 weight percent Radix Codonopsis, about 3 weight percent Fructus Rubi, about 10 weight percent Radix Astragali, about 8 weight percent Rhizoma Dioscoreae, about 3 weight percent Radix Paeoniae Alba, about 7 weight percent Herba Ecliptae, about 8 weight percent Fructus Mori, about 3 weight percent Radix Curcumae, and about 3 weight percent Herba Cynomorii.

11. The composition of claim 10, wherein the composition consists of a plurality of herbal ingredients prepared through processing methods, extraction methods, and purification methods.

12. The composition of claim 11, wherein the processing methods include at least one member selected from the group consisting of: washing, steaming, extracting, roasting, herb frying, salt frying, honey frying, wine frying, earth frying, vinegar frying, calcining drying, heating, and grinding.

13. The composition of claim 12, wherein the extraction methods include at least one member selected from the group consisting of: soaking, heating, steaming, evaporation, compressing, and solvent extraction with at least one of the following: water, alcoholic solvents mixed with water, petroleum ether, hexane, diethyl amine, diethyl ether, cyclohexane, tert-butyl alcohol, isopropanol, acetonitrile, aceton, ethanol, methyl isobutyl ketone, isobutyl alcohol, 1-propanol, methyl ethyl ketone, 2-butanol, isoamyl alcohol, 1-butanol, diethyl ketone, 1-octanol, p-xylene, m-xylene, toluene, dimethoxyethane, benzene, butyl acetate, 1-chlorobutane, tetrahydrofuran, ethyl acetate, o-xylene, hexamethylphosphorus triamide, 2-ethoxyethyl ether. N,N-dimethylacetamide, diethylene glycol dimethyl ether, N,N-dimethylformamide, 2-methoxyethanol, pyridine, propanoic acid, 2-methoxyethyl acetate, benzonitrile, 1-Methyl-2-pyrrolidinone, hexamethylphosphoramide, 1,4-dioxane, acetic acid, acetic anhydride, dimethyl sulfoxide, chlorobenzene, ethylene glycol, diethylene glycol, propylene carbonate, formic acid, 1,2-dichloroethane, glycerin, carbon disulfide, 1,2-dichlorobenzene, methylene chloride, nitromethane, 2,2,2-trifluoroethanol, chloroform, 1,1,2-trichlorotrifluoroethane, carbon tetrachloride, and tetrachloroethylene.

14. The composition of claim 13, wherein the purification methods include at least one member selected from the group consisting of: column separation, solvent partition, evaporation, and solvent precipitation.

15. The composition of claim 10, wherein Fructus Lycii, the goji berry, is the fruit of the plant Lycium barbarum L.; Semen Cuscutae , the dodder seed, is the seed of the plant Cuscuta chinensis Lam.; Radix Salviae Miltiorrhizae, the red sage root, is the root and rhizome of the plant Salvia miltiorrhiza Bge.; Pericarpium Citri Reticulatae Viride preparata, the processed green orange peel, unripe tangerine peel, an immature mandarin orange peel, is the rind of green fruit of the plant Citrus reticulate Blanco; Radix Polygoni Multiflori preparata, the processed fleece flower root or Chinese knotweed, is the processed root of the plant Polygonum multiflorum Thunb.; Herba Epimedii, the horny goat weed, is the arial part of the plant Epimedium brevicornum Maxim., Epimedium sagittatum (Sieb. Et Zucc.) Maxim., Epimedium pubescens Maxim., Epimedium wushanense T. S. Ying, Epimedium koreanum Nakai; Radix Codonopsis, the codonopsis root, is the root of the plant Codonopsis pilosula (Franch) Nannf., Codonopsis pilosula Nannf. var. moderta (Nannf.) L., Codonopsis tangshen Oliv.; Fructus Rubi, the raspberry, is the fruit of the plant Rubus chingii Hu; Radix Astragali, the astragalus root is the root of the plant Astragalus membranaceus (Fisch.) Bge. var. mongholicus (Bge.) Hsiao, Astragalus membranaceus (Fisch.) Bge.; Rhizoma Dioscoreae, the Chinese yam, is the rhizome from plant Dioscorea opposite Thunb.; Radix Paeoniae Alba, the white peony root, is the root without bark of the plant Paeonia lactiflora Pall.; Herba Ecliptae, the false daisy, is the arial part of the plant Eclipta prostrata L.; Fructus Mori, the mulberry, is the fruit of the plant Morus alba L.; Radix Curcumae, the curcuma tuber, is the root and rhizome of the plant Curcuma wenyujin Y. H. Chen et C. Ling, Curcuma kwangsiensis S. Lee et C. F. Liang, Curcuma longa L., Curcuma phaeocaulis Val; Herba Cynomorii, the cynomorium is the fleshy stem of the plant Cynomorium songaricum Rupr,.

16. An herbal composition for enhancing fertility in males and females, the composition consisting of:
About 8 weight percent Fructus Lycii, about 3 weight percent Semen Cuscutae, about about 3 weight percent Radix Salviae Miltiorrhizae, about 5 weight percent, Pericarpium Citri Reticulatae Viride preparata, about 5 weight percent Radix Polygoni Multiflori preparata, about 10 weight percent Herba Epimedii, about 5 weight percent Radix Codonopsis, about 15 weight percent Fructus Rubi, about 3 weight percent Radix Astragali, about 3 weight percent Rhizoma Dioscoreae, about 10 weight percent Radix Paeoniae Alba, about 10 weight percent Herba Ecliptae, about 5 weight percent Fructus Mori, about 5 weight percent Radix Curcumae, and about 5 weight percent Herba Cynomorii.

17. The composition of claim 16, wherein the composition consists of a plurality of herbal ingredients prepared through processing methods, extraction methods, and purification methods.

18. The composition of claim 17, wherein the processing methods include at least one member selected from the group consisting of: washing, steaming, extracting, roasting, herb frying, salt frying, honey frying, wine frying, earth frying, vinegar frying, calcining drying, heating, and grinding.

19. The composition of claim 18, wherein the extraction methods include at least one member selected from the group consisting of: soaking, heating, steaming, evaporation, compressing, and solvent extraction with at least one of the following: water, alcoholic solvents mixed with water, petroleum ether, hexane, diethyl amine, diethyl ether, cyclohexane, tert-butyl alcohol, isopropanol, acetonitrile, aceton, ethanol, methyl isobutyl ketone, isobutyl alcohol, 1-propanol, methyl ethyl ketone, 2-butanol, isoamyl alcohol, 1-butanol, diethyl ketone, 1-octanol, p-xylene, m-xylene, toluene, dimethoxyethane, benzene, butyl acetate, 1-chlorobutane, tetrahydrofuran, ethyl acetate, o-xylene, hexamethylphosphorus triamide, 2-ethoxyethyl ether. N,N-dimethylacetamide, diethylene glycol dimethyl ether, N,N-dimethylformamide, 2-methoxyethanol, pyridine, propanoic acid, 2-methoxyethyl acetate, benzonitrile, 1-Methyl-2-pyrrolidinone, hexamethylphosphoramide, 1,4-dioxane, acetic acid, acetic anhydride, dimethyl sulfoxide, chlorobenzene, ethylene glycol, diethylene glycol, propylene carbonate, formic acid, 1,2-dichloroethane, glycerin, carbon disulfide, 1,2-dichlorobenzene, methylene chloride, nitromethane, 2,2,2-trifluoroethanol, chloroform, 1,1,2-trichlorotrifluoroethane, carbon tetrachloride, and tetrachloroethylene.

20. The composition of claim 16, wherein Fructus Lycii, the goji berry, is the fruit of the plant Lycium barbarum L.; Semen Cuscutae, the dodder seed, is the seed of the plant Cuscuta chinensis Lam.; Radix Salviae Miltiorrhizae, the red sage root, is the root and rhizome of the plant Salvia miltiorrhiza Bge.; Pericarpium Citri Reticulatae Viride preparata, the processed green orange peel, unripe tangerine peel, an immature mandarin orange peel, is the rind of green fruit of the plant Citrus reticulate Blanco; Radix Polygoni Multiflori preparata, the processed fleece flower root or Chinese knotweed, is the processed root of the plant Polygonum multiflorum Thunb.; Herba Epimedii, the horny goat weed, is the arial part of the plant Epimedium brevicornum Maxim., Epimedium sagittatum (Sieb. Et Zucc.) Maxim., Epimedium pubescens Maxim., Epimedium wushanense T. S. Ying, Epimedium koreanum Nakai; Radix Codonopsis, the codonopsis root, is the root of the plant Codonopsis pilosula (Franch) Nannf., Codonopsis pilosula Nannf. var. moderta (Nannf.) L., Codonopsis tangshen Oliv.; Fructus Rubi, the raspberry, is the fruit of the plant Rubus chingii Hu; Radix Astragali, the astragalus root is the root of the plant Astragalus membranaceus (Fisch.) Bge. var. mongholicus (Bge.) Hsiao, Astragalus membranaceus (Fisch.) Bge.; Rhizoma Dioscoreae, the Chinese yam, is the rhizome from plant Dioscorea opposite Thunb.; Radix Paeoniae Alba, the white peony root, is the root without bark of the plant Paeonia lactiflora Pall.; Herba Ecliptae, the false daisy, is the arial part of the plant Eclipta prostrata L.; Fructus Mori, the mulberry, is the fruit of the plant Morus alba L.; Radix Curcumae, the curcuma tuber, is the root and rhizome of the plant Curcuma wenyujin Y. H. Chen et C. Ling, Curcuma kwangsiensis S. Lee et C. F. Liang, Curcuma longa L., Curcuma phaeocaulis Val; Herba Cynomorii, the cynomorium is the fleshy stem of the plant Cynomorium songaricum Rupr.
